(19) United States
(12) Patent Application Publication
(10) Pub. No.: US 2024/0016644 A1
(43) Pub. Date: Jan. 18, 2024

(54) BUNION SPLINT ASSEMBLY WITH ADJUSTABLE SUPPORT CUSHION

(71) Applicant: Scholl's Wellness Company LLC, Parsippany, NJ (US)

(72) Inventor: Edmund Farmer, Seymour, CT (US)

(21) Appl. No.: 18/220,545

(22) Filed: Jul. 11, 2023

Related U.S. Application Data
(60) Provisional application No. 63/388,596, filed on Jul. 12, 2022.

(51) Int. Cl. A61F 5/01

(52) U.S. Cl. CPC A61F 5/019

(57) ABSTRACT

Embodiments of the present disclosure relate to a bunion splint assembly comprising a flexible body shaped to conform to a human foot, an elongated rigid member disposed within fabric layers of the flexible body, and a ring-shaped cushion sized to encompass a bunion on the big toe that is adapted to be removably secured to an inner surface of the flexible body.

### CROSS-REFERENCE TO RELATED APPLICATION(S)

This application claims the benefit of priority of U.S. Provisional Patent Application No. 63/388,596, filed on Jul. 12, 2022, the disclosures of which are hereby incorporated by reference herein in their entireties.

### BACKGROUND

A bunion is an inflammation of the bursa that normally cushions the metatarsophalangeal (MTP) joint. When the big toe moves toward the second toe, the inner half of the bursa can become pinched causing the outer half to bulge with the appearance of a bone-like projection. This inflammation can be sensitive to the touch, especially in the center of the bulge. Treatments for bunions often include pharmaceutical approaches such as the use of non-steroidal anti-inflammatory drugs (NSAIDs) and other anti-inflammatory drugs. Other approaches utilize mechanical structures that apply pressure to realign the toe while providing a cushion to surround the bunion. However, these approaches fail to facilitate optimal placement of the cushion, which can exacerbate the inflammation.

### SUMMARY

The following summary presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect, a bunion splint assembly comprises: a flexible body shaped to conform to a human foot; an elongated rigid member disposed within fabric layers of the flexible body such that, when the flexible body is secured to the human foot, the elongated rigid member is aligned along the side of the human foot corresponding to the location of the big toe and applies pressure in a direction toward the second toe; and a ring-shaped cushion sized to encompass a bunion on the big toe, the ring-shaped cushion being adapted to be removably and adjustably secured to an inner surface of the flexible body.

In at least one embodiment, when the flexible body is secured to the human foot, the ring-shaped cushion is disposed between the elongated rigid member and the big toe and engages a region of the big toe surrounding the bunion without applying force to a center portion of the bunion.

In at least one embodiment, the ring-shaped cushion is adapted to removably couple to a treatment site-engaging surface of the inner surface of the flexible body.

In at least one embodiment, the elongated rigid member is disposed between the treatment site-engaging surface and an opposing outer surface of the bunion splint assembly.

In at least one embodiment, each of the treatment site-engaging surface and the opposing outer surface comprise a foam material.

In at least one embodiment, the elongated rigid member is permanently disposed between the treatment site-engaging surface and the opposing outer surface.

In at least one embodiment, the elongated rigid member is removable from between the treatment site-engaging surface and the opposing outer surface.

In at least one embodiment, the ring-shaped cushion comprises a fastening material affixed thereto, and the fastening material is adapted to reversibly secure the ring-shaped cushion to the treatment site-engaging surface.

In at least one embodiment, the ring-shaped cushion has a tapered outer diameter that is largest at an interface with the fastening material.

In at least one embodiment, the ring-shaped cushion comprises a polymer gel or a foam material.

In at least one embodiment, the ring-shaped cushion defines an elongated aperture having a first inner diameter that is greater than an orthogonal second inner diameter, and the first inner diameter is from about 2.5 cm to about 5 cm.

In at least one embodiment, the elongated rigid member comprises a substantially flat metal bar.

In at least one embodiment, the bunion splint assembly further comprises a plurality of straps adapted to secure the flexible body to the human foot when worn.

In at least one embodiment, a first strap of the plurality of straps is adapted to wrap at least partially around the big toe and be reversibly secured to an outer surface of the flexible body when worn.

In at least one embodiment, a second strap and a third strap of the plurality of straps are disposed along a common edge of the flexible body and are adapted to reversibly engage each other and secure the flexible body to the heel when worn.

In at least one embodiment, a fourth strap of the plurality of straps is adapted to be reversibly secured to the outer surface of the flexible body on a top portion of the foot when worn. In at least one embodiment, the inner surface is formed from a different material than the outer surface, and the material of the outer surface is adapted to reversibly engage a hook-and-loop fastener material of the plurality of straps.

In a further aspect, a kit comprises: any of the aforementioned bunion splint assemblies; and one or more additional ring-shaped cushions. In at least one embodiment, each ring-shaped cushion has a different sized aperture.

In a further aspect, a kit comprises: a pair of any of the aforementioned bunion splint assemblies. In at least one embodiment, a first bunion splint assembly is adapted for conforming to a left foot and a second bunion splint assembly is adapted for conforming to a right foot.

In a further aspect, a method of treating a bunion comprises applying any of the aforementioned bunion splint assemblies to a human foot on which the bunion is present.

In at least one embodiment, the method further comprises adjusting a position of the ring-shaped cushion on the flexible body to control a location of engagement of the ring-shaped cushion with the human foot relative to the bunion.

### BRIEF DESCRIPTION OF THE DRAWINGS

FIG. 1A shows an outer surface of an exemplary bunion splint assembly designed for the left foot in accordance with at least one embodiment.

FIG. 1B shows an inner surface of the exemplary bunion splint assembly designed for the left foot in accordance with at least one embodiment.

FIG. 2A shows an outer surface of an exemplary bunion splint assembly designed for the right foot in accordance with at least one embodiment.

FIG. 2B shows an inner surface of the exemplary bunion splint assembly designed for the right foot in accordance with at least one embodiment.

FIG. 3A shows a top-down view of an exemplary ring-shaped cushion and fastening material in accordance with at least one embodiment.

FIG. 3B shows a side view of the exemplary ring-shaped cushion and fastening material in accordance with at least one embodiment.

FIG. 3C shows a blown-up view of the exemplary ring-shaped cushion and fastening material in accordance with at least one embodiment.

FIG. 4 illustrates a method of applying the bunion splint assembly to a human foot in accordance with at least one embodiment.

FIG. 5 illustrates adjustment of a ring-shaped cushion in accordance with at least one embodiment.

FIG. 6A shows a first perspective view of an exemplary bunion splint assembly when worn on a right foot in accordance with at least one embodiment.

FIG. 6B shows a second perspective view of the exemplary bunion splint assembly when worn on the right foot in accordance with at least one embodiment.

### DETAILED DESCRIPTION

A bunion splint assembly with an adjustable support cushion is described herein. A cushioning ring can be used as a support cushion to surround the bunion without contacting the sensitive projected area. This allows for the avoidance of pressure on the bunion that would occur if a bandage was used, and protects the bunion from be subjected to added pressure that can exacerbate the inflammation. Proper placement of the ring-shaped cushion around the bunion is important for ensuring that no pressure is exerted on a central portion of the bunion by the protective ring or by other components of the bunion splint assembly. As each individual has a different distance of the MTP joint to the end of the big toe, the position of the protective cushion or ring should be adjusted to improve the effectiveness in alleviating bunion pain.

Certain embodiments of the present disclosure address these problems and others by utilizing a moveable and repositionable support cushion (e.g., a ring-shaped cushion). The embodiments advantageously reduce bunion inflammation and pain by aligning the big toe for increased comfort and correction without applying pressure to sensitive portions of the bunion. The bunion splint assembly can be a universal size that fits and adjusts to all foot shapes.

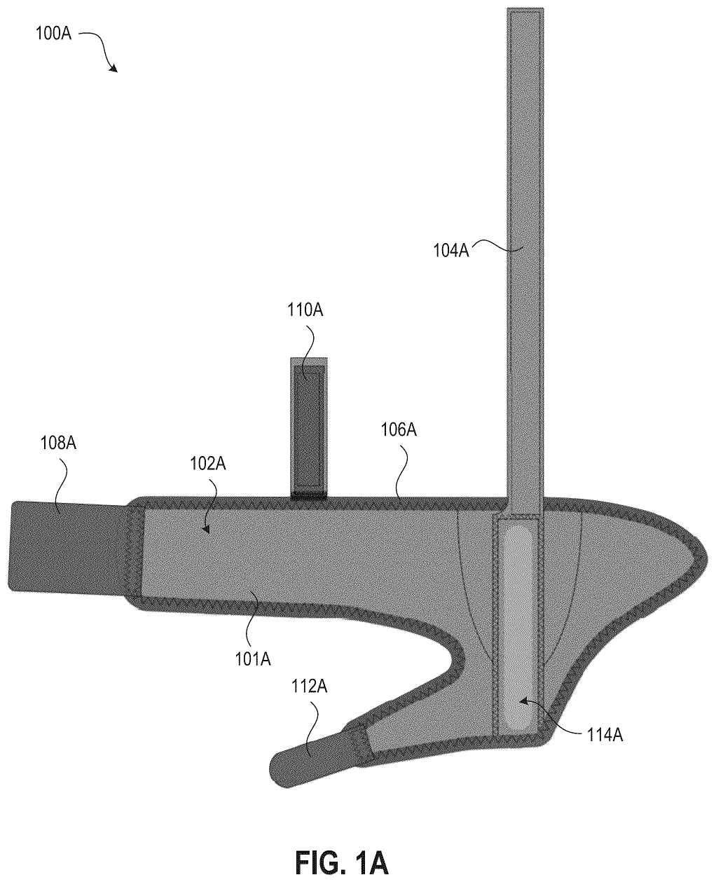

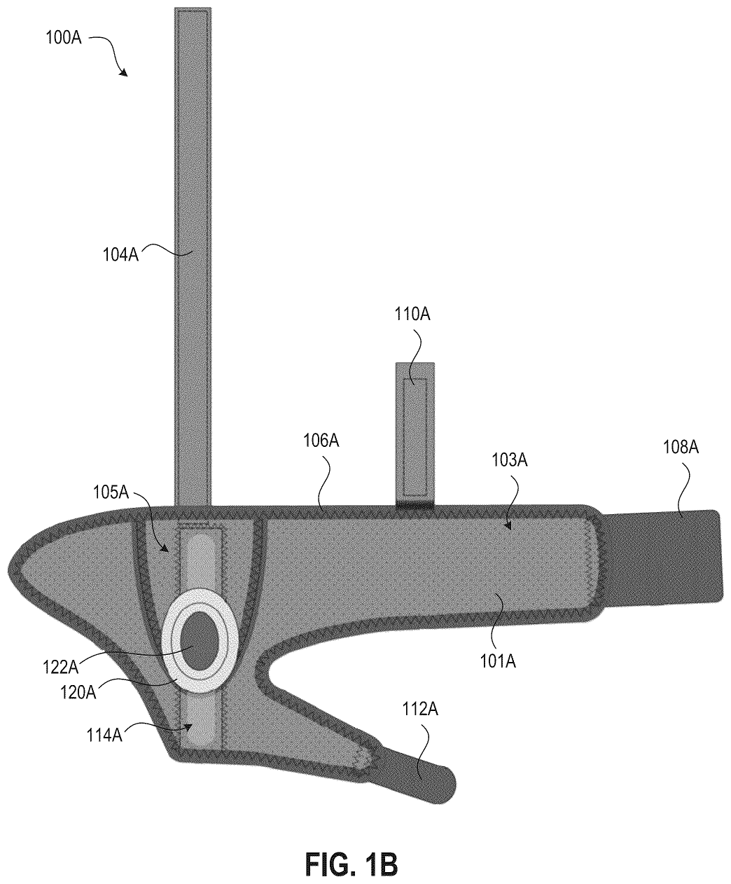

FIGS. 1A and 1B show an outer surface 102A and an inner surface 103A, respectively, of an illustrative bunion splint assembly 100A designed for the left foot in accordance with at least one embodiment. The bunion splint includes a flexible body 101A shaped to conform to the foot and a plurality of straps 104A, 108A, 110A, and 112A disposed along an outer seam 106A of the flexible body 101A that secure the flexible body 101A in place when worn. In at least one embodiment, an elongated rigid member 114A, such as a substantially flat metal bar, is disposed within fabric layers (e.g., foam layers) between the outer surface 102A and the inner surface 103A of the flexible body 101A so as to not contact the skin of the wearer. When the flexible body 101A is secured to the foot, the elongated rigid member 114A (with its the shape being illustrated despite being obscured by the fabric of the outer surface 102A and the inner surface 103A) is aligned along the side of the foot corresponding to the location of the big toe. In at least one embodiment, the elongated rigid member 114A is permanently disposed between the treatment site-engaging surface 105A and the opposing outer surface 102A (e.g., the two surfaces are sewn together). In at least one embodiment, the elongated rigid member 114A is removable from between the treatment site-engaging surface 105A and the opposing outer surface 102A, for example, via a slot or pocket. For example, in at least one embodiment, metal bar can be removed and replaced with a material of different stiffness/thickness as desired.

In FIG. 1B, a ring-shaped cushion 120A is shown affixed to the inner surface 103A of the flexible body 101A at a treatment site-engaging surface 105A, which is above the elongated rigid member 114A. In at least one embodiment, the ring-shaped cushion 120A is affixed to one side of a fastening material 122A (e.g., a pad), while the opposing side of the fastening material 122A is adapted to removably couple to the treatment site-engaging surface 105A, for example, via a hook-and-loop engagement, an adhesive, or any other suitable mechanism for reversible attachment.

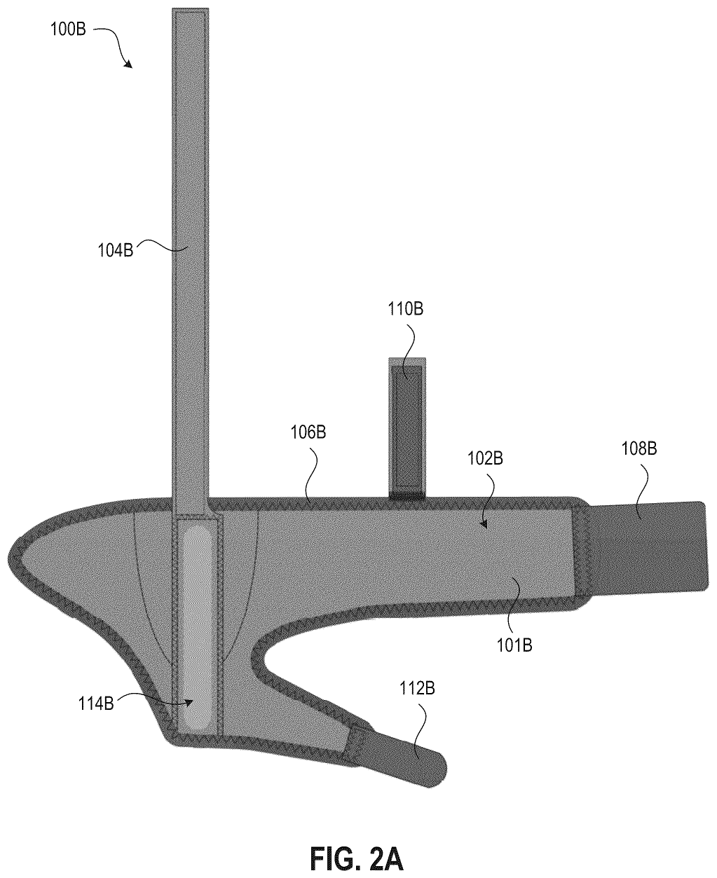

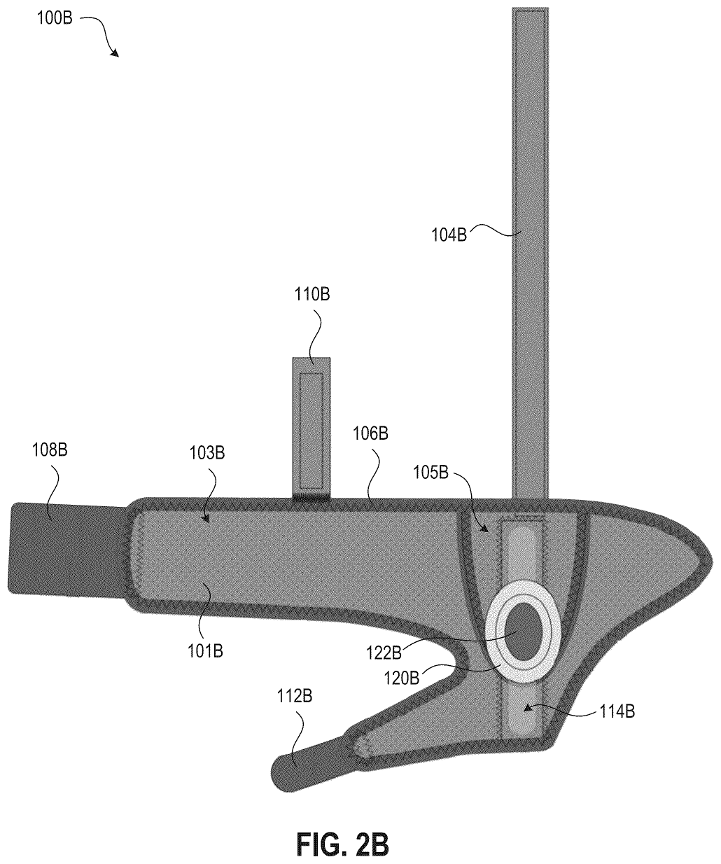

FIGS. 2A and 2B show a similar bunion split assembly 100B designed for the right foot, which is effectively a mirror image of the bunion splint assembly 100A. Similarly labeled components labeled as “B” in FIGS. 2A and 2B correspond to the components labeled as “A” in FIGS. 1A and 1B.

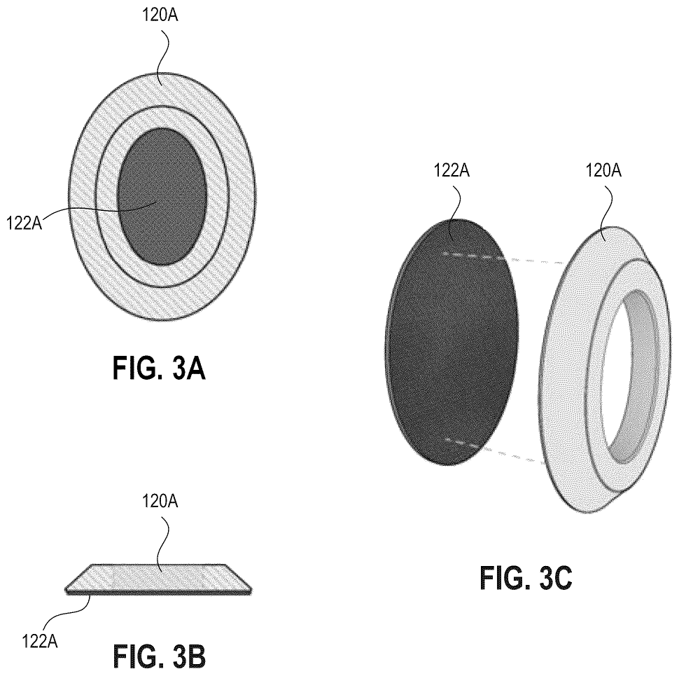

FIGS. 3A, 3B, and 3C show top-down, side, and blown-up views, respectively, of the exemplary ring-shaped cushion 120A and fastening material 122A. In at least one embodiment, the ring-shaped cushion 120A has a tapered outer diameter that is largest at the interface with the fastening material 122A, as illustrated in FIG. 3B. In at least one embodiment, the ring-shaped cushion 120A comprises a polymer gel or a foam material. In at least one embodiment, the ring-shaped cushion 120A defines an elongated aperture having a first inner diameter that is greater than an orthogonal second inner diameter, and wherein the first inner diameter is from about 2.5 cm to about 5 cm.

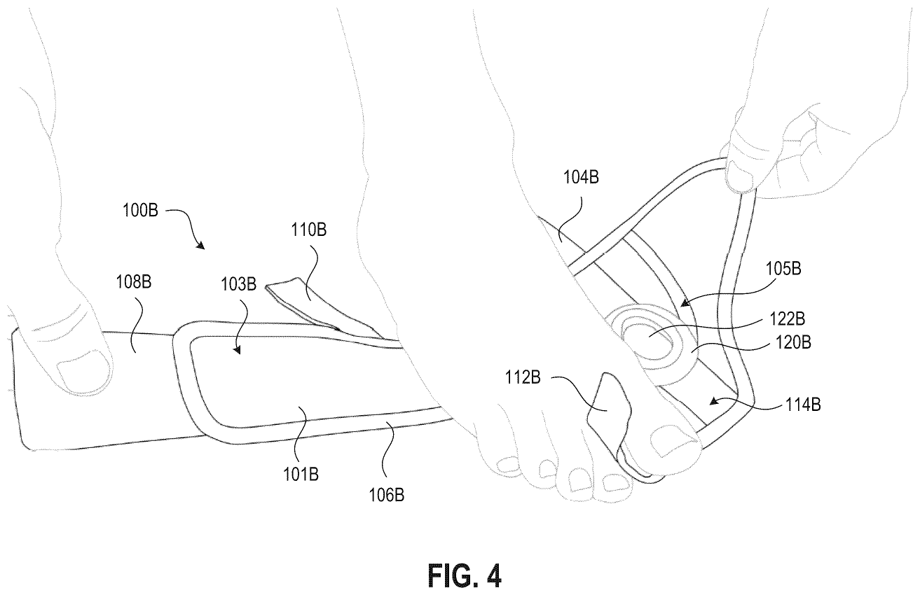

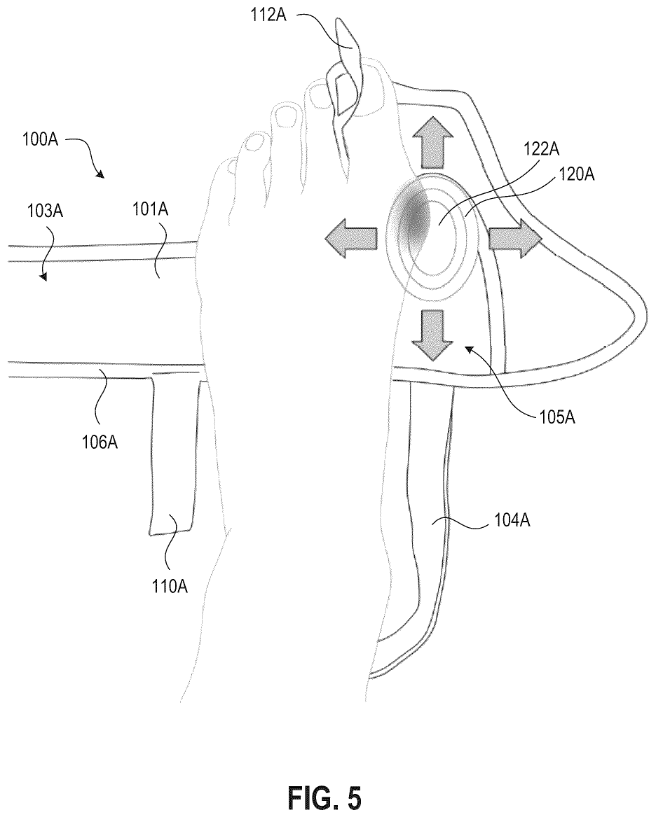

FIG. 4 illustrates a method of applying the bunion splint assembly 100B to a left human foot in accordance with at least one embodiment. As shown, the wearer stands on the inner surface 103B of the flexible body 101B while wrapping a big toe fastener around the inside of the big toe and affixing the strap 112B. Before wrapping the foot, the ring-shaped cushion 122B can be positioned on the treatment site-engaging surface 105B so that it will surround the outside of the bunion when engaged, illustrated in FIG. 5. Once wrapped around the foot and secured thereto, the ring-shaped cushion 120B encompasses the bunion and redistributes force exerted by the elongated rigid member 114B around bunion without the ring-shaped cushion 120B or another portion of the bunion splint assembly 100B applying a force to the center portion of the bunion.

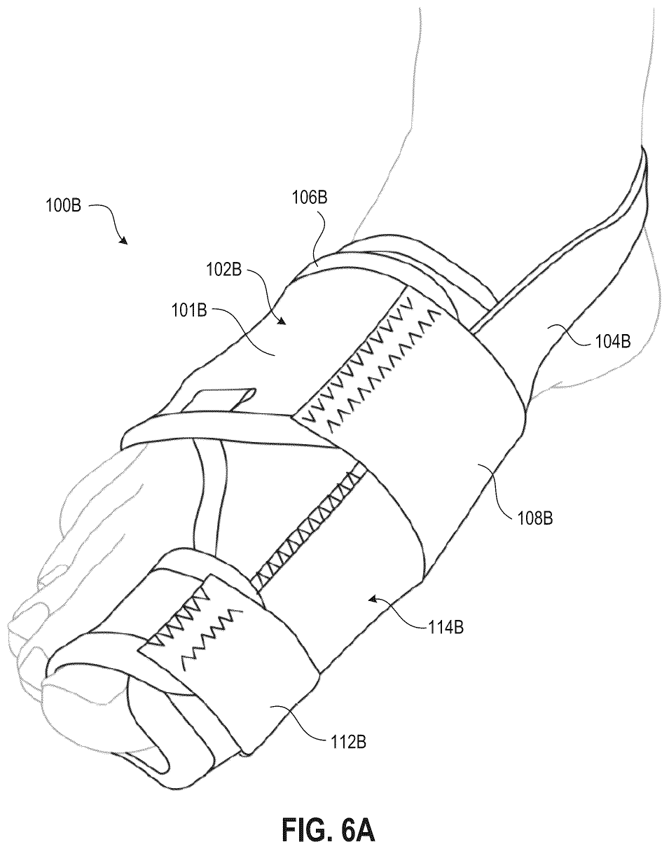

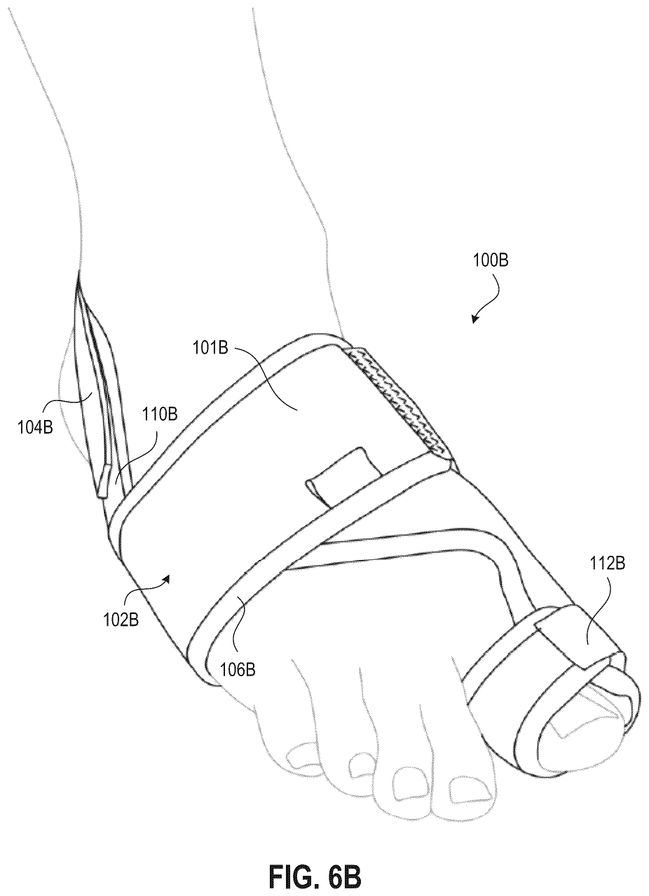

FIGS. 6A and 6B show different perspective views of the bunion splint assembly 100B when worn on a right human foot in accordance with at least one embodiment, which illustrate the placement of straps to secure the bunion splint assembly 100B to the foot. In at least one embodiment, the strap 112B is adapted to wrap at least partially around the big toe and be reversibly secured to an outer surface 102B of the flexible body 101B. In at least one embodiment, the straps 104B and 110B are disposed along a common edge (e.g., the seam 106B) of the flexible body 101B and are adapted to reversibly engage each other and secure the flexible body 101B to the heel. In at least one embodiment, the strap 108B is adapted to be reversibly secured to the outer surface 102B of the flexible body 101B on a top portion and/or side portion of the foot. In at least one embodiment, the inner surface 103B (not shown in FIGS. 6A and 6B) is formed from a different material than the outer surface 102B. For example, the inner surface 103B may be formed from a material such as a polyblend stretch terry cloth fabric, or other suitable material that avoids irritating the skin during prolonged contact. The outer surface 102B may be formed, for example, from a stretch polyester knap fabric, or other suitable material adapted to reversibly engage a hook-and-loop fastener material of the straps.

In at least one embodiment, a kit is contemplated, which may include one or more or more bunion splint assemblies as described herein. For example, the kit includes a single bunion splint assembly for each of the left foot and the right foot. In at least one embodiment, a kit may include additional ring-shaped cushions having different sized apertures (e.g., 1-inch longest diameter, 1.5-inch longest diameter, 2-inch longest diameter, 2.5-inch longest diameter, 3-inch longest diameter, etc., or within any range defined therebetween).

It is to be understood by those of ordinary skill in the art that the bunion splint assemblies depicted herein are merely illustrative of suitable devices used in connection with the various methods described herein. Other variations may be utilized, including those for which one or more components are omitted or added.

In the foregoing description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure. For simplicity of explanation, the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment” or “one embodiment” means that a particular feature, structure, or characteristic described in connection with the embodiment is included in at least one embodiment. Thus, the appearances of the phrase “an embodiment” or “one embodiment” in various places throughout this specification are not necessarily all referring to the same embodiment.

As used herein, the term “about” is used to describe and account for small fluctuations. For example, “about” may mean the numeric value may be modified by ±1%, ±0.5%, ±0.4%, ±0.3%, ±0.2%, ±0.1%, or ±0.05%. All numeric values are modified by the term “about” whether or not explicitly indicated. Numeric values modified by the term “about” include the specific identified value. For example, “about 5.0” includes 5.0.

The present disclosure is not to be limited in scope by the specific embodiments described herein. Indeed, other various embodiments of and modifications to the present disclosure, in addition to those described herein, will be apparent to those of ordinary skill in the art from the description and accompanying drawings. Thus, such other embodiments and modifications are intended to fall within the scope of the present disclosure. Further, while the present disclosure has been described in the context of a particular embodiment in a particular environment for a particular purpose, those of ordinary skill in the art will recognize that its usefulness is not limited thereto and that the present disclosure may be beneficially implemented in any number of environments for any number of purposes. Accordingly, the embodiments set forth below should be construed in view of the full breadth and spirit of the present disclosure as described herein, along with the full scope of equivalents.

What is claimed is:

1. A bunion splint assembly comprising:
a flexible body shaped to conform to a human foot;
an elongated rigid member disposed within fabric layers of the flexible body such that, when the flexible body is secured to the human foot, the elongated rigid member is aligned along the side of the human foot corresponding to the location of the big toe and applies pressure in a direction toward the second toe; and
a ring-shaped cushion sized to encompass a bunion on the big toe, the ring-shaped cushion being adapted to be removably and adjustably secured to an inner surface of the flexible body.

2. The bunion splint assembly of claim 1, wherein, when the flexible body is secured to the human foot, the ring-shaped cushion is disposed between the elongated rigid member and the big toe and engages a region of the big toe surrounding the bunion without applying force to a center portion of the bunion.

3. The bunion splint assembly of claim 1, wherein the ring-shaped cushion is adapted to removably couple to a treatment site-engaging surface of the inner surface of the flexible body.

4. The bunion splint assembly of claim 3, wherein the elongated rigid member is disposed between the treatment site-engaging surface and an opposing outer surface of the bunion splint assembly.

5. The bunion splint assembly of claim 4, wherein each of the treatment site-engaging surface and the opposing outer surface comprise a foam material.

6. The bunion splint assembly of claim 4, wherein the elongated rigid member is permanently disposed between the treatment site-engaging surface and the opposing outer surface.

7. The bunion splint assembly of claim 4, wherein the elongated rigid member is removable from between the treatment site-engaging surface and the opposing outer surface.

8. The bunion splint assembly of claim 7, wherein the ring-shaped cushion comprises a fastening material affixed thereto, wherein the fastening material is adapted to reversibly secure the ring-shaped cushion to the treatment site-engaging surface.

9. The bunion splint assembly of claim 8 wherein the ring-shaped cushion has a tapered outer diameter that is largest at an interface with the fastening material.

10. The bunion splint assembly of claim 1, wherein the ring-shaped cushion comprises a polymer gel or a foam material.

11. The bunion splint assembly of claim 1, wherein the ring-shaped cushion defines an elongated aperture having a first inner diameter that is greater than an orthogonal second inner diameter, and wherein the first inner diameter is from about 2.5 cm to about 5 cm.

12. The bunion splint assembly of claim 1, wherein the elongated rigid member comprises a substantially flat metal bar.

13. The bunion splint assembly of claim 1, further comprising a plurality of straps adapted to secure the flexible body to the human foot when worn.

14. The bunion splint assembly of claim 13, wherein a first strap of the plurality of straps is adapted to wrap at least partially around the big toe and be reversibly secured to an outer surface of the flexible body when worn.

15. The bunion splint assembly of claim 14, wherein a second strap and a third strap of the plurality of straps are disposed along a common edge of the flexible body and are adapted to reversibly engage each other and secure the flexible body to the heel when worn.

16. The bunion splint assembly of claim 15, wherein a fourth strap of the plurality of straps is adapted to be reversibly secured to the outer surface of the flexible body on a top portion of the foot when worn, and wherein the inner surface is formed from a different material than the outer surface, and wherein the material of the outer surface is adapted to reversibly engage a hook-and-loop fastener material of the plurality of straps.

17. A kit comprising:
the bunion splint assembly of claim 1; and
one or more additional ring-shaped cushions, wherein each ring-shaped cushion has a different sized aperture.

18. A kit comprising:
a pair of bunion splint assemblies according to claim 1, wherein a first bunion splint assembly is adapted for conforming to a left foot and a second bunion splint assembly is adapted for conforming to a right foot.

19. A method of treating a bunion comprising applying the bunion splint assembly of claim 1 to a human foot on which the bunion is present.

20. The method of claim 19, the method further comprises adjusting a position of the ring-shaped cushion on the flexible body to control a location of engagement of the ring-shaped cushion with the human foot relative to the bunion.
